(19) United States
(12) Patent Application Publication
(10) Pub. No.: US 2025/0352215 A1
(43) Pub. Date: Nov. 20, 2025

(54) VASCULAR OCCLUSION COILS AND OCCLUDERS FORMED THEREWITH

(71) Applicant: VenaCore Inc., Willmington, DE (US)

(72) Inventors: Michael Gabriel TAL, Tel Aviv (IL); Nir HOLTZMAN, Rishon Lezion (IL); Amit GREENER, Tel-Aviv (IL)

(21) Appl. No.: 19/194,548

(22) Filed: Apr. 30, 2025

Related U.S. Application Data
(63) Continuation of application No. PCT/US2023/078387, filed on Nov. 1, 2023.
(60) Provisional application No. 63/421,722, filed on Nov. 2, 2022.

(51) Int. Cl. A61B 17/12; A61B 17/00

(52) U.S. Cl. CPC A61B 17/12145; A61B 17/12031; A61B 17/12109; A61B 2017/00862; A61B 2017/1205

(57) ABSTRACT

Vascular occluders may comprise containers defining a container space. The container space may receive a vascular occlusion coil. The vascular occlusion coil may have an elastically relaxed configuration comprising a series of arced segments arranged in two or more different orientations. The vascular occlusion coil may comprise a cylindrical spiral body with a core member provided therein. The vascular occlusion coil may transition from a secondary structure to a tertiary structure as it is dispensed into the container space.

### RELATED APPLICATIONS

This application is a continuation of International Application PCT/US2023/078387, filed Nov. 1, 2023, which application claims priority to U.S. Provisional Application 63/421,722 entitled Forming a Vascular Occluder in a Blood Vessel filed on Nov. 2, 2022.

### FIELD

The present disclosure relates to vascular implants and methods for implanting vascular implants in a blood vessel, and more particularly, but not exclusively, to systems, devices and methods for forming a vascular occluder in blood vessels.

### BACKGROUND

Some medical procedures, such as embolization, involve occluding a blood vessel such as for reducing pressure on aneurysms, restricting a hemorrhage, or diminishing blood supply to tumors or growths in the body.

Vascular occlusion coils may be used for occluding voids in a patient vasculature using endovascular coiling and embolization techniques. Such coils have a minute spiral body usually made of soft metal and are sized and configured for delivery and implantation using a catheter. One or more coils are delivered in a single site, then manually curled and packed together at the target implantation site until forming a plug-like structure which serves to harvest coagulated blood adhering to its outer surface for gradually causing local occlusion and embolization.

Vascular plugs are a different type of mechanical embolization device commonly used for occluding a targeted portion of vein or artery with a relatively low-profile delivery and can be released in a controlled fashion. Some vascular plugs are considered advantageous to coils in such respects, however they are substantially more expensive than the metal coils and less adaptable for implantation in target implantation sites varying in size and shape.

In light of these and other disadvantages, there is need for improved vascular occlusion coils, and for improved systems and methods for deploying and implanting vascular occlusion coils in bodily lumens, for achieving improved results in one or more of: reducing time and/or skills required for delivering, deploying, manipulating and/or packing coils into self-anchoring structures configured for occluding open-ended bodily lumens; reducing overall volume and/or total surface area of implanted metal in patient's body; and predetermined or self-conformable coil-based solution independent of local conditions and landing zone shape and dimensions of the target implantation site.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

The present disclosure relates to vascular implants and methods for implanting vascular implants in a blood vessel, and more particularly, but not exclusively, to systems, devices and methods for forming a vascular occluder in a blood vessel.

In one embodiment, a vascular occlusion coil has an elastically relaxed configuration comprising a series of arced segments arranged in two or more different orientations. The vascular occlusion coil is configured to be elastically deformed into a linear configuration for placement in a coil dispensing catheter, and the vascular occlusion coil is configured to sequentially form the series of arced segments of the elastically relaxed configuration as it is pushed out from a distal end of the coil dispensing catheter. The vascular occlusion coil may be used in a variety of procedures and is especially advantageously used in occluding lumens of blood vessels by being dispensed into a container positioned in a target blood vessel to be occluded.

In one embodiment, a vascular occluder comprises a container comprising a side wall, wherein the side wall defines a container space. A vascular occlusion coil is in the container space. Arced segments of the vascular occlusion coil are positioned and pushed against different portions of the side wall, wherein at least some different arced segments extend along distinct planes having different spatial orientation relative to planes along which other arced segments extend, thereby collectively forming a structure in the container space configured to resist compressive forces extending along multiple spatial directions.

In certain embodiments, there is provided a vascular occluder forming system, which can comprise: (a) a vascular occlusion coil, (b) a container comprising a fluid permeable side wall enclosing a container space configured for implantation within a tubular lumen section of a target blood vessel, and (c) a coil dispenser configured to release the vascular occlusion coil into the container space. When the vascular occlusion coil is released into the container, arced segments of the vascular occlusion coil are sequentially positioned and pushed against different portions of the side wall. In some embodiments, the vascular occlusion coil may comprise a cylindrical spiral body enclosing an elongated coil passage, and a core member provided within the cylindrical spiral body and configured to force the vascular occlusion coil into a secondary structure wherein the cylindrical spiral body is bent, rotated and/or twisted into a three-dimensional form.

In some embodiments, the container is configured to accommodate into the container space the vascular occlusion coil in the secondary structure, and to force the vascular occlusion coil into a tertiary structure derivable from the side wall when the cylindrical spiral body is axially compacted and/or radially expanded from the secondary structure.

In some embodiments, the vascular occlusion coil in the tertiary structure is configured to anchor the container to the target blood vessel in the tubular lumen section.

In some embodiments, the flexible side wall extends between a proximal end and a distal end, encloses the container space, and the container is capable of expanding and/or compressing radially and/or axially and is configured to engage an inner wall surface of the target blood vessel with an outer surface of the side wall, to resist flow of blood passing through the side wall, and/or to accumulate coagulated blood in the container space.

In some embodiments, the outer surface of the side wall is configured with a variable average roughness changeable in accordance with relative radial and/or axial compression of the container, when implanted in the target blood vessel.

In some embodiments, the variable average roughness is greater when the container contains the vascular occlusion coil in the tertiary structure than when the container is vacant, for anchoring the container to the target blood vessel.

In some embodiments, the side wall includes a first material configured with a first roughness and a second material configured with a second roughness greater than the first roughness, wherein when the container is axially extended and/or radially compressed relative to an elastically relaxed form thereof the outer surface contains a greater ratio between accumulated surface area of the first material and of the second material than when the container is axially compressed and/or radially extended relative to the elastically relaxed form thereof.

In some embodiments, the side wall is meshed, woven, braided or perforated.

In some embodiments, the side wall is formed of metallic material such as Ni—Ti alloy, optionally in a form of braided wire.

In some embodiments, the core member is extendable along the elongated coil passage when in an elastically stretched form, and, when released from the elastically stretched form to a more elastically relaxed configuration, the core member is configured to force the vascular occlusion coil into the secondary structure.

In some embodiments, the vascular occlusion coil in the secondary structure is elastically extendable to a substantially linear stretched form configured for fitting and advancing by way of pushing in a microcatheter lumen.

In some embodiments, the core member includes an elastic wire or strand optionally of metallic material.

In some embodiments, the vascular occlusion coil in the secondary structure forms a plurality of helical segments interlinked with a corresponding curved linking portion.

In some embodiments, the helical segments are substantially coinciding and spaced apart with each other, such that they form a tubular-like frame configured with a substantially constant helix diameter and/or pitch.

In some embodiments, each one of the helical segments is configured to form one of the arced segments, when fully released in the container space.

In some embodiments, at least one of the helical segments includes a single winding or a partial winding of the core member between each sequential pair of the corresponding curved linking portion.

In some embodiments, at least one of the curved linking portions is formed as an arc, optionally having an arc central angle within a range of about 45° to about 225°, optionally particularly greater than about 90°, optionally particularly about 180°.

In some embodiments, the helical segments in at least one sequential pair of the helical segments are oppositely winded relative to each other.

In some embodiments, at least a portion of the core member is axially slidable within the elongated coil passage.

In some embodiments, a distal portion of the vascular occluding coil extends distally relative to a distal end of the core member.

In some embodiments, the coil dispenser is configured to allow gradual continuous release of the vascular occlusion coil in the container space while restraining a portion of the vascular occlusion coil in the stretched form before the vascular occlusion coil is fully released in the container space.

In some embodiments, the vascular occluder forming system is configured to form a vascular occluder in a target blood vessel, such that, when vascular occlusion coil is released from the coil dispenser in the container space, arced segments of the vascular occlusion coil in the secondary structure are sequentially positioned and pushed against different portions of the tubular wall, wherein each one of the arced segment extends along a distinct plane having different spatial orientation relative to planes along which other arced segments extend, thereby collectively form a cocoon-like inner structure configured to resist compressive forces extending along multiple spatial directions.

In some embodiments, the coil dispenser is a microcatheter or a distal portion of, or is connected to, a microcatheter.

In some embodiments, the coil dispenser is detachably connected or connectable to the container.

In certain embodiments, there is provided a method for forming a vascular occluder in a blood vessel, the method comprising providing a coil dispenser connected to a container, the container comprising a flexible tubular wall extending between a proximal end and a distal end and enclosing a container space, the container is capable of expanding radially and/or compressing axially and is configured to resist flow of blood passing therethrough and to accumulate coagulated blood in the container space, positioning the container in a target blood vessel, such that the container engages an inner wall surface of the target blood vessel, and releasing a vascular occluding coil, gradually, in the container space thereby allowing the vascular occluding coil to elastically reshape from a substantially linear stretched form in the filler dispenser to a three-dimensional frame structure in the container.

In some embodiments, the releasing includes sequentially positioning and pushing arced segments of the vascular occluding coil against different portions of the tubular wall, wherein each one of the arced segments extends along a distinct plane having different spatial orientation relative to planes along which other arced segments extend, until collectively forming an inner structure configured to resist compressive forces extending along multiple spatial directions.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIGS. 1A-1D schematically illustrate exemplary scenarios representing steps in a method for forming a vascular occluder in a blood vessel using a vascular occlusion coil, according to some embodiments;

FIGS. 2A-2E schematically illustrate exemplary scenarios representing steps in an exemplary procedure for forming a vascular occluder in a blood vessel, according to some embodiments;

FIGS. 3A-3C schematically illustrate, respectively, a vascular occlusion coil, a core member insertable to the vascular occlusion view, and the vascular occlusion coil in a secondary structure following insertion of the core member thereinto, according to some embodiments;

FIGS. 4A-4B illustrate views of an exemplary vascular occlusion coil provided in an exemplary secondary structure, according to some embodiments.

### DETAILED DESCRIPTION

The following description and examples illustrate some e exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Certain embodiments relate to vascular implants and methods for implanting vascular implants in a blood vessel, and more particularly, but not exclusively, to systems, devices and methods for forming a vascular occluder in blood vessels. In some embodiments, there is provided a system for forming a vascular occluder that includes a vascular occlusion coil and means for deploying and shaping the coil in a tubular lumen of a target blood vessel. In some embodiments, such means may include a core member configured to shape (e.g., impose a shape of) the vascular occlusion coil from an inner passage or space enclosed by the spiral body of the vascular occlusion coil, and/or a shaped or shapeable container configured to shape (e.g., impose a shape of) the vascular occlusion coil from its surrounding space formed by the container, to which the coil is inserted.

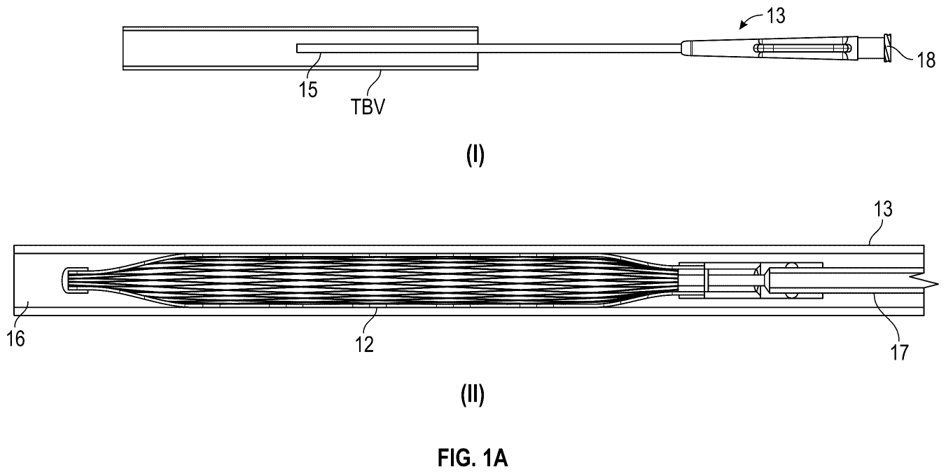

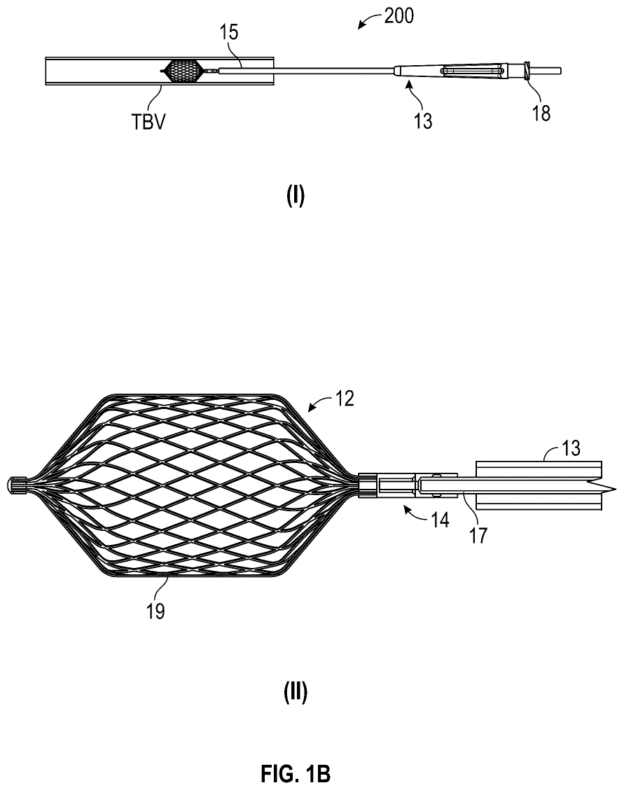

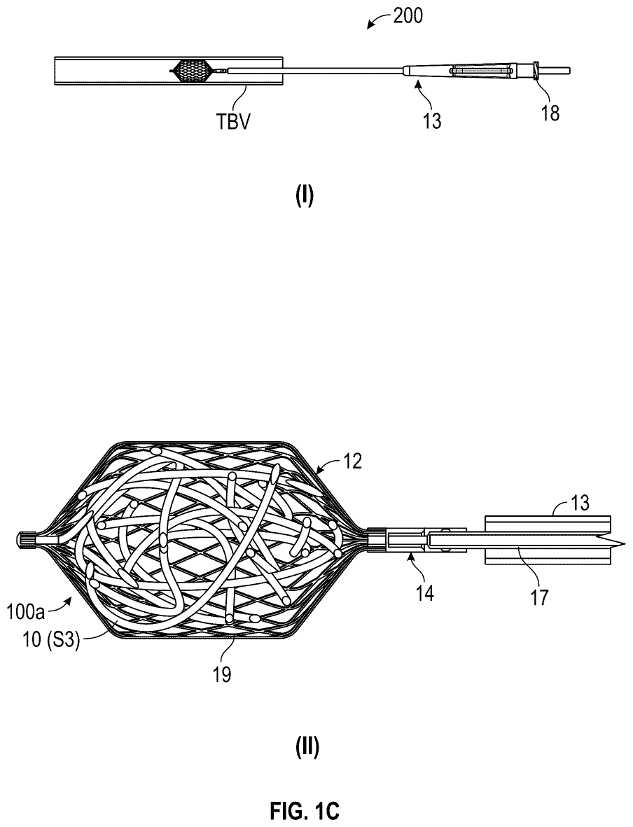

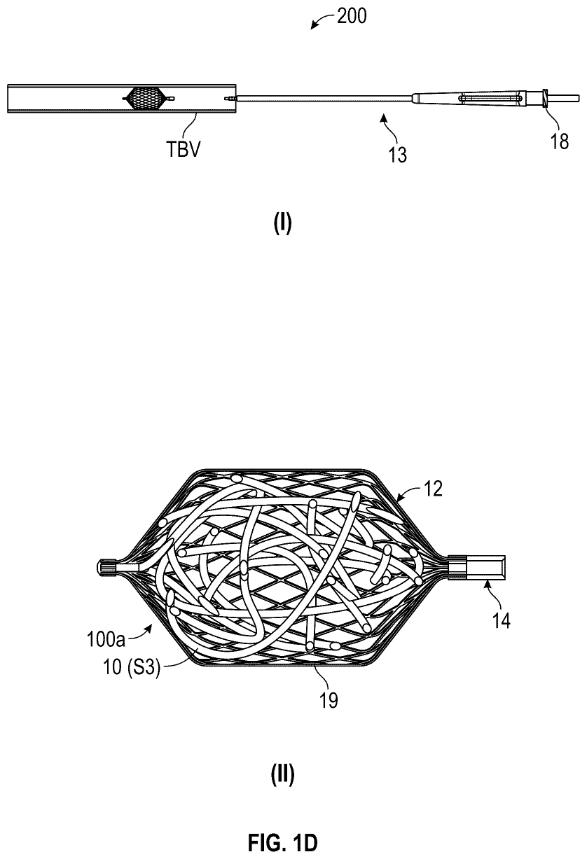

FIGS. 1A-1D schematically illustrate exemplary scenarios representing steps in a method for forming a vascular occluder 100a in a target blood vessel TBV using a vascular occlusion coil 10. A system 200 for forming vascular occluder 100a includes coil 10, a container 12, a catheter 13 (e.g., a single-lumen catheter, optionally a microcatheter), and a coil dispenser 14.

FIG. 1A(I) shows a first scenario wherein catheter 13 with a distal portion 15 thereof is provided in a lumen of target blood vessel (TBV) (catheter 13 is shown in a side view, and blood vessel TBV is shown in a side cross-sectional view for ease of description). FIG. 1A(II) illustrates catheter distal portion 15 in an enlarged side cross-sectional view. Container 12 is provided and/or deliverable in a crimped or radially compacted configuration in a lumen 16 of catheter 13 (optionally particularly in distal portion 15). Container 12 is pushable and optionally releasably connected to an elongated pusher 17 (such as by way of threading or snap-locking) which optionally extends along lumen 16 such that a proximal end thereof is manipulatable by a user via a proximal portion 18 of catheter 13.

FIG. 1B(I) shows a second scenario wherein container 12 is pushed via distal portion 15 into the lumen of target blood vessel TBV (container 12 and catheter 13 are shown in side view, and blood vessel TBV is shown in a side cross-sectional view for ease of description). FIG. 1B(II) illustrates container 12 connected to pusher 17 emerging via distal portion 15 in an enlarged side cross-sectional view. Container 12 is allowed to elastically expand creating a contained space, optionally until engaging a side wall 19 thereof with inner wall surface of target blood vessel TBV. In some embodiments, the user can choose container 12 of a specific size and/or shape in accordance with some fitting consideration thereof in target blood vessel TBV. Container 12 may be formed as a tubular or other shaped structure by way of one or more wires which may be braided or otherwise arranged and coupled as is known in the art, optionally metal wires (e.g., Ni—Ti or Co—Cr alloy wires), although it can be made by other materials, optionally non-stretching wires, such as nylon, polyester, cotton, polypropylene or aramid. In some embodiments, container 12 is configured such that it cannot be effectively inflated by gas and/or liquid, and/or that it is configured to self-expand while allowing fluid flow thereinto.

FIG. 1C(I) shows a third scenario wherein vascular occlusion coil 10 is completely dispensed within container 12 (container 12 and catheter 13 are shown in side view, and blood vessel TBV is shown in a side cross-sectional view for ease of description). FIG. 1C(II) illustrates container 12 connected to pusher 17 emerging via distal portion 15 in an enlarged side cross-sectional view. Vascular occlusion coil 10 is arranged into a three-dimensional tertiary structure (which may be cocoon-like) denoted S3 in FIG. 1C(II) by being pushed gradually through coil dispenser 14 with arced segments thereof engaging the shaped covering of container 12 forcing it to deform as such. Coil 10 may be advanced using pusher 17 or through pusher 17, optionally using other means. In some embodiments, once fully deployed in container 12, vascular occlusion coil 10 forces container 12 to expand laterally and/or compress axially, thereby increasing anchoring force or pressure against walls of target blood vessel TBV. FIGS. 1D(I) and (II) similarly illustrate system 200 after vascular occluder 100a, formed by container 12 filled with coil 10 in the tertiary structure, is disconnected from pusher 17 and after catheter 13 is removed from blood vessel TBV, leaving vascular occluder in place.

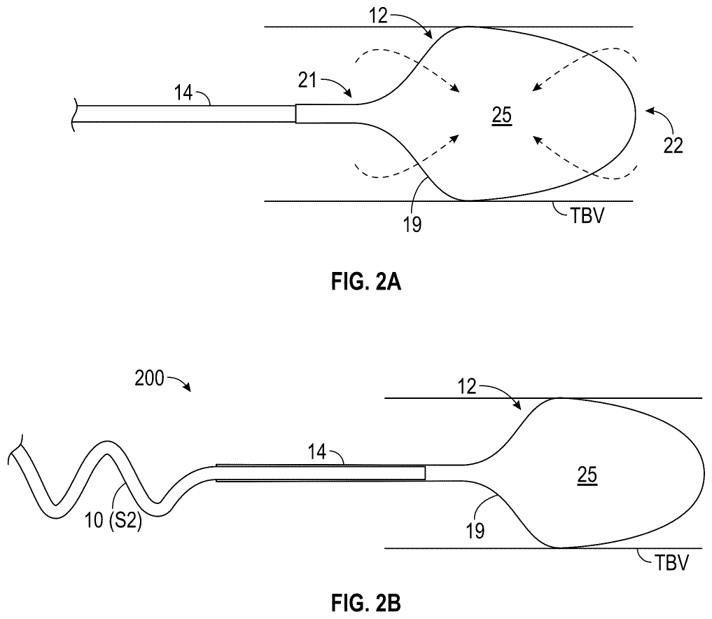

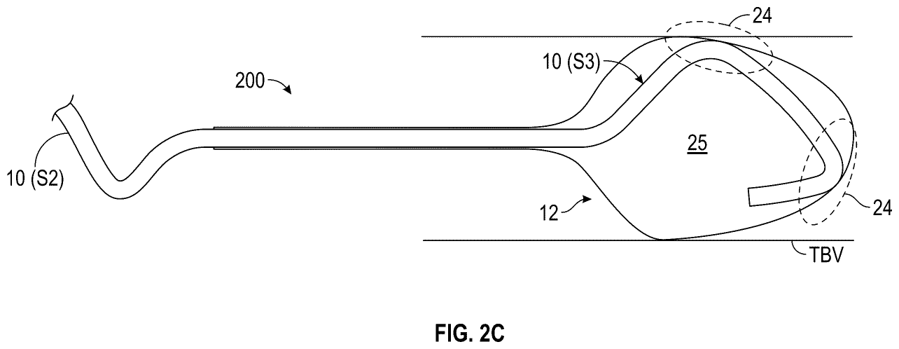

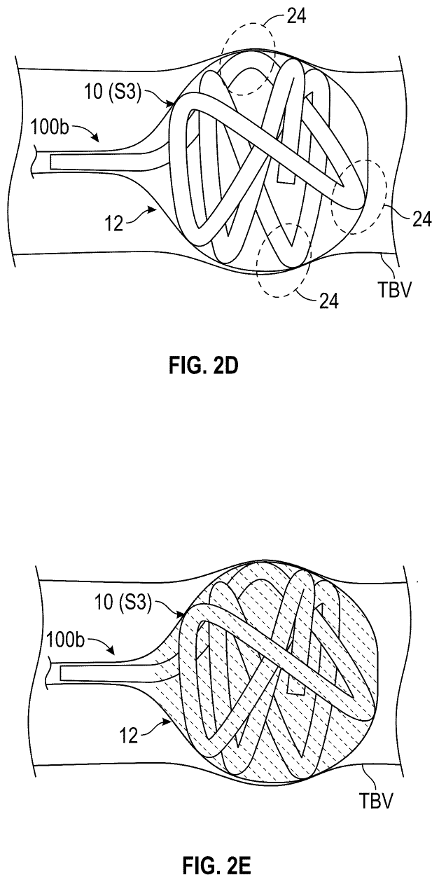

FIGS. 2A-2E schematically illustrate exemplary scenarios representing steps in an exemplary procedure for forming a vascular occluder 100b in a target blood vessel TBV using a vascular occluder forming system 200 comprising vascular occlusion coil 10 and a container 16. As above, container 16 includes a fluid permeable shaped covering 19 enclosing a container space or contained space 25 configured for implantation within a tubular lumen section of target blood vessel TBV. Container 16 is configured to accommodate into container space 25 vascular occlusion coil 10 in a secondary structure S2, and to force the vascular occlusion coil into a tertiary structure S3 which is configured to anchor the container 16 to the target blood vessel in the tubular lumen section. The tertiary structure S3 is derivable from the shape of the side wall when the container 12 is axially compacted and/or radially expanded by the coil 10. System 200 may also include a coil dispenser 14 configured to allow gradual continuous release of vascular occlusion coil 10 in container space 18 while restraining a portion of vascular occlusion coil 10 in a stretched and/or linear form before vascular occlusion coil 10 is fully released within container space 18. Coil dispenser 14 may be a part or a portion of container 16 or it may be a separate member fixedly or detachably connected thereto. In some other embodiments, coil dispenser 14 may be a portion of a catheter or microcatheter or may be provided in a lumen thereof, and/or it may be fixedly or detachably connected thereto.

Shaped covering 19 includes a flexible tubular wall extending between a proximal end 21 and a distal end 22 and enclosing contained space 25. Container 12 is capable of expanding and/or compressing radially and/or axially and is configured to engage an inner wall surface of target blood vessel TBV with an outer surface of the side wall 19, to resist flow of blood passing through the tubular wall, and/or to accumulate coagulated blood in the contained space 25. In some embodiments, at least the outer surface of the side wall 19 is configured with a variable average roughness changeable in accordance with relative radial and/or axial compression of the container, when implanted in target blood vessel TBV. In some embodiments, the variable average roughness is greater when container 12 contains vascular occlusion coil 10 in the tertiary structure S3 in the contained space 25 than when the contained space 25 of container 12 is vacant, for improved anchoring of the container 12 to the target blood vessel TBV.

For example, the side wall 19 may include a first material (e.g., metal) configured with a first roughness and a second material (e.g., polymer) configured with a second roughness greater than the first roughness. In some such embodiments, when container 12 is axially extended and/or radially compressed, relative to an elastically relaxed form thereof, outer surface of side wall 19 contains a greater ratio between accumulated surface area of the first material and of the second material than when container 12 is axially compressed and/or radially extended relative to the elastically relaxed form thereof. In some embodiments, the side wall 19 is formed of one or more braided or woven metal threads or filaments covered with at least partially fluid permeable polymeric membrane.

FIG. 2A shows container 12 after positioning thereof in target blood vessel TBV, with side wall 19 engaging inner surface of blood vessel TBV. Container 12 may be inserted into target blood vessel TBV in a compacted form and then actively (e.g., selectively by a user) or passively (e.g., elastically) expanded to engage the blood vessel wall. As shown, side wall 19 is fluid permeable and allows inflow of blood from lumen of blood vessel TBV surrounding container 12 through the side wall 19 into contained space 25 (as schematically illustrated with dashed arrows). FIG. 2B shows the blood-filled container 12 during advancement of vascular occlusion coil 10 into container 12 through coil dispenser 14.

FIG. 2C shows system 200 when vascular occlusion coil 10 is partially released in container space 18, and FIG. 2D shows system 200 when coil 10 is mostly or fully released in contained space 25 and, optionally, after coil dispenser 14 is removed, thereby forming vascular occluder 100b. As shown, system 200 is configured such that, when vascular occlusion coil 10 is released from coil dispenser 14 in contained space 25, arced segments 24 of vascular occlusion coil 10 in the secondary structure S2 are sequentially positioned and pushed against different portions of the side wall 19 of container 12, wherein each one of the arced segment 24 extends along a distinct plane having different spatial orientation relative to planes along which other arced segments extend. As such, the arced segments 24 collectively form tertiary structure S3 as an inner structure (which may be cocoon-like) configured to resist compressive forces extending along multiple spatial directions. Vascular occlusion coil 10 in the tertiary structure S3 may cause container 12 to enlarge radially and compress axially thereby facilitating anchoring to target blood vessel TBV. FIG. 2E shows vascular occluder 100 after the blood entrapped in contained space 25 is substantially coagulated, solidifies, and/or adheres to vascular occluding coil 10 and side wall 19, thereby sufficiently (e.g., advantageously completely or essentially completely in some embodiments) blocking lumen of target blood vessel TBV.

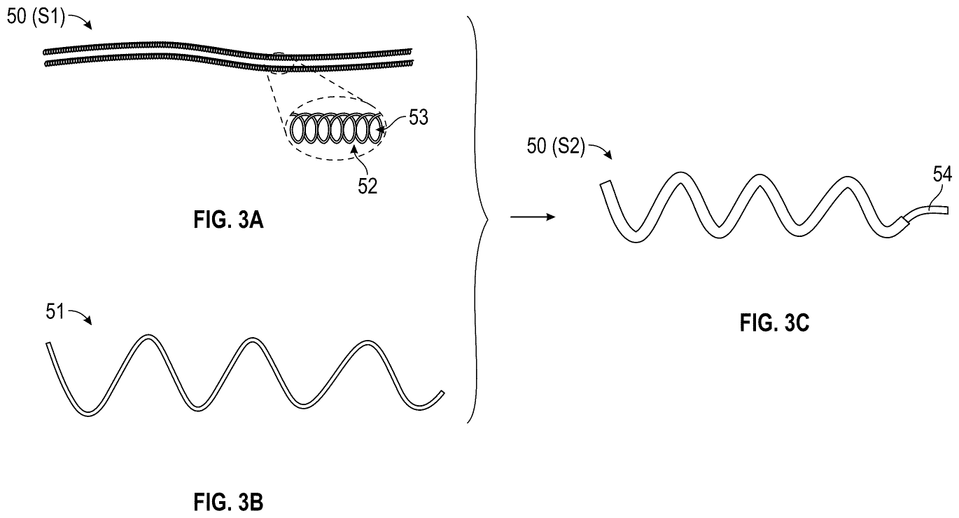

FIGS. 3A-3C schematically illustrate, respectively, a vascular occlusion coil 50 that may be used in the vascular occluder embodiments described above. In this embodiment, vascular occlusion coil 50 has a primary structure S1 (shown in FIG. 3A), a core member 51 (shown in FIG. 3B), and a secondary structure (shown in FIG. 3C) following insertion of core member 51 thereinto. Vascular occlusion coil 50 in its primary structure S1 includes a cylindrical spiral body 52 enclosing an elongated coil passage 53. Coil 50 is optionally flexible and/or malleable; additionally, or alternatively, coil 50 may be elastic with tendency to form a substantially straight shape when in an elastically relaxed configuration.

Core member 51 is readily provided or is insertable within cylindrical spiral body 52 and (when provided therein) is configured to force vascular occlusion coil 50 into a secondary structure S2 wherein cylindrical spiral body 52 is shaped in a three-dimensional form, optionally particularly bent, rotated and/or twisted form. Core member 51 is extendable along elongated coil passage 53 when in an elastically stretched form. When in the secondary structure S2, vascular occlusion coil 50 is elastically extendable to a substantially linear stretched form configured for fitting and advancing by way of pushing in a microcatheter lumen such as catheter 13 above. When released from the elastically stretched form to a more elastically relaxed configuration, core member 51 is configured to force vascular occlusion coil 50 into the secondary structure S2. Core member 51 may be unconnected and/or axially slidable within elongated coil passage 53. Alternatively, core member 51 may be connected or connectable to vascular occlusion coil 50 in at least one portion. In some embodiments, a proximal portion of core member 51 is connected to a proximal portion of vascular occlusion coil 50 whereas a distal end of core member is free to move axially relative to a distal portion 54 of vascular occlusion coil 50. As shown in FIG. 3C, distal portion 54 may extend distally relative to a distal end of core member 51.

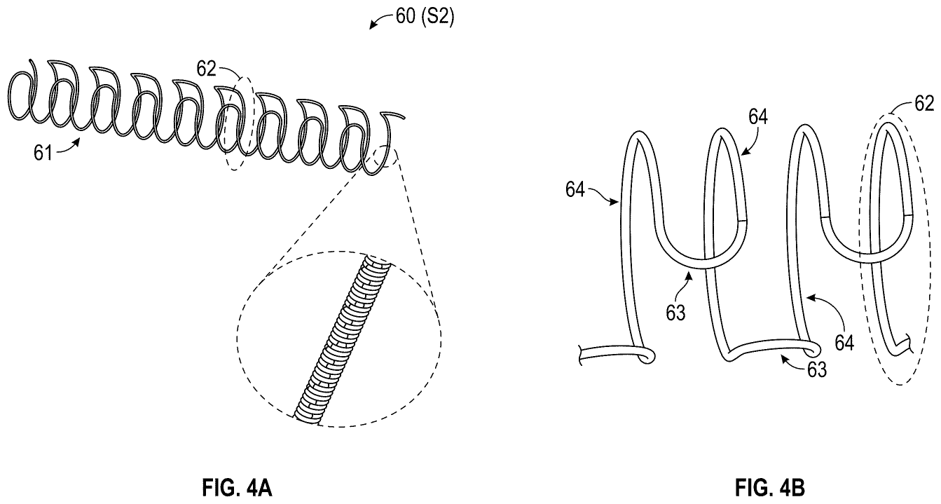

FIGS. 4A-4B illustrate views of an exemplary vascular occlusion coil 60 provided in an exemplary secondary structure S2, optionally after being internally shaped using a core member (e.g., core member 51). FIG. 10A shows vascular occlusion coil 50 in an elastically relaxed configuration, and a magnified or ‘zoomed-in’ portion thereof illustrating its primary coiled structure. FIG. 4B shows a portion of section of coil 60 with a few helical segments thereof.

As shown, vascular occlusion coil 60 includes a cylindrical spiral body 61 and forms an elastically relaxed state a secondary structure S2 wherein the cylindrical spiral body 61 is bent, rotated and/or twisted into a three-dimensional form. Vascular occlusion coil 60 in the secondary structure S2 is elastically extendable to a substantially linear stretched form configured for fitting and advancing by way of pushing in a microcatheter lumen and/or a coil dispenser.

Vascular occlusion coil 60 in the secondary structure forms a plurality of helical segments 62 interlinked with a corresponding curved linking portion 63. Helical segments 62 are substantially coinciding and spaced apart with each other, such that they form a tubular-like frame configured with a substantially constant helix diameter and/or pitch. Each helical segment 62 is configured to form an arced segment (e.g., similar to arced segments 24) when fully released in a container space (e.g., container space 25). At least one helical segment 62 includes a single or a partial winding 64 between each sequential pair of the corresponding curved linking portion 63. At least one curved linking portion 63 is formed as an arc, optionally having an arc central angle within a range of about 45° to about 225°, optionally particularly greater than about 90°, optionally particularly about 180°. The helical segments in at least one sequential pair of helical segments 62 are optionally oppositely winded relative to each other.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

When applied to a numerical value, the term ‘about’, as used herein, refers to +10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

1. A vascular occluder forming system comprising:
a vascular occlusion coil;
a container comprising a fluid permeable side wall enclosing a container space configured for implantation within a tubular lumen section of a target blood vessel, wherein the container space is configured to accommodate the vascular occlusion coil;
a coil dispenser configured to release the vascular occlusion coil into the container space;
wherein the coil dispenser, the container, and the vascular occlusion coil are configured such that when the vascular occlusion coil is released from the coil dispenser into the container, arced segments of the vascular occlusion coil are sequentially positioned and pushed against different portions of the side wall.

2. The vascular occluder forming system of claim 1, wherein at least some different arced segments extend along distinct planes having different spatial orientation relative to planes along which other arced segments extend, thereby collectively forming a structure in the container space configured to resist compressive forces extending along multiple spatial directions.

3. The vascular occluder forming system of claim 2, wherein the vascular occlusion coil comprises a cylindrical spiral body enclosing an elongated coil passage.

4. The vascular occluder forming system of claim 3, wherein the vascular occlusion coil comprises a core member within the cylindrical spiral body.

5. The vascular occluder forming system of claim 4, wherein the core member comprises an elastic wire.

6. The vascular occluder forming system of claim 5, wherein the core member is formed of metal.

7. The vascular occluder forming system of claim 4, wherein the core member is configured to force the vascular occlusion coil into a secondary structure in which the cylindrical spiral body is bent, rotated and/or twisted into a three-dimensional form.

8. The vascular occluder forming system of claim 7, wherein the container is configured to receive into the container space the vascular occlusion coil in the secondary structure and to force the vascular occlusion coil into a tertiary structure derivable from a shape of the side wall when the cylindrical spiral body is axially compacted and/or radially expanded from the secondary structure when released into the container space from the coil dispenser.

9. The vascular occluder forming system of claim 1, wherein the vascular occlusion coil has an elastically relaxed configuration comprising series of arced segments arranged in two or more different orientations, wherein the vascular occlusion coil is configured to be elastically deformed into a linear configuration for placement in the coil dispenser, and wherein the vascular occlusion coil is configured to sequentially form the series of arced segments of the elastically relaxed configuration as it is pushed out from a distal end of the coil dispenser.

10-22. (canceled)

23. A vascular occlusion coil comprising:
a cylindrical spiral body enclosing an elongated coil passage; and
a core member positioned within the elongated coil passage.

24. The vascular occlusion coil of claim 23, wherein the core member is configured to force the vascular occlusion coil into a secondary structure in which the cylindrical spiral body is bent, rotated and/or twisted into a three-dimensional form.

25. The vascular occlusion coil of claim 24, wherein the secondary structure comprises a series of arced segments arranged in two or more different orientations.

26. A vascular occlusion coil having an elastically relaxed configuration comprising a series of arced segments arranged in two or more different orientations, wherein the vascular occlusion coil is configured to be elastically deformed into a linear configuration for placement in a coil dispensing catheter, and wherein the vascular occlusion coil is configured to sequentially form the series of arced segments of the elastically relaxed configuration as it is pushed out from a distal end of the coil dispensing catheter.

27. The vascular occlusion coil of claim 26, wherein the vascular occlusion coil comprises:
a cylindrical spiral body enclosing an elongated coil passage; and
a core member positioned within the elongated coil passage.

28. The vascular occlusion coil of claim 26, wherein the vascular occlusion coil forms a plurality of helical segments interlinked with a corresponding curved linking portion.

29. The vascular occlusion coil of claim 28, wherein the helical segments are substantially coinciding and spaced apart with each other, such that they form a tubular-like frame configured with a substantially constant helix diameter and/or pitch.

30. The vascular occlusion coil of claim 28, wherein each one of the helical segments is configured to form one of the arced segments.

31. The vascular occlusion coil of claim 28, wherein at least one of the helical segments includes a single winding or a partial winding between each sequential pair of the corresponding curved linking portion.

32. The vascular occlusion coil of claim 28, wherein at least one of the curved linking portions is formed as an arc having an arc central angle within a range of about 45° to about 225°, optionally particularly greater than about 90°, optionally particularly about 180°.

33. The vascular occlusion coil of claim 28, wherein at least one of the curved linking portions is formed as an arc having an arc central angle greater than 90°.

34. The vascular occlusion coil of claim 28, wherein at least one of the curved linking portions is formed as an arc having an arc central angle of about 180°.

35. The vascular occlusion coil of claim 28, wherein the helical segments in at least one sequential pair of the helical segments are oppositely winded relative to each other.

36-58. (canceled)
